(19) United States
(12) Patent Application Publication
(10) Pub. No.: US 2004/0030209 A1
(43) Pub. Date: Feb. 12, 2004

(54) Method for the production of alkyl aryl sulphonates

(76) Inventors: Thomas Narbeshuber, Mannheim (DE); Ulrich Steinbrenner, Neustadt (DE); Gerhard Krack, Limburgerhof (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/432,361

(22) PCT Filed: Nov. 16, 2001
(86) PCT No.: PCT/EP01/13322

(30) Foreign Application Priority Data
Nov. 30, 2000 (DE) 100 59 398.4

(51) Int. Cl. C07C 1/00; C07C 2/00; C11D 17/00

(52) U.S. Cl. 585/323; 510/424

(57) ABSTRACT

The preparation of alkylaryl compounds takes place by

1) preparation of a mixture of, on statistical average, predominantly monobranched C₁₀₋₁₄-olefins by

a) reaction of a C₄-olefin mixture over a metathesis catalyst for the preparation of an olefin mixture comprising 2-pentene and/or 3-hexene, and optional removal of 2-pentene and/or 3-hexene, followed by dimerization of the resulting 2-pentene and/or 3-hexene over a dimerization catalyst to give a mixture comprising C₁₀₋₁₂-olefins, and optionally removal of the C₁₀₋₁₂-olefins, or

b) extraction of predominantly monobranched paraffins from kerosene cuts and subsequent dehydrogenation, or

c) Fischer-Tropsch synthesis of olefins or paraffins, where the paraffins are dehydrogenated, or

d) dimerization of shorter-chain internal olefins, or

e) isomerization of linear olefins or paraffins, where the isomerized paraffins are dehydrogenated,

2) reaction of the olefin mixture obtained in stage 1) with an aromatic hydrocarbon in the presence of an alkylation catalyst which contains zeolites of the faujasite type.

The present invention relates to processes for the preparation of alkylaryl compounds and alkylarylsulfonates, to alkylaryls and alkylarylsulfonates obtainable by these processes, to the use of said alkylaryl compounds and alkylarylsulfonates as surfactants, preferably in detergents and cleaners, and to detergents and cleaners comprising these alkylaryl compounds and alkylarylsulfonates.

Alkylbenzenesulfonates (ABS) have been used for a long time as surfactants in detergents and cleaners. Following the use initially of such surfactants based on tetrapropylenebenzenesulfonate, which, however, had poor biodegradability, alkylbenzenesulfonates which are as linear as possible (LAS) have since been prepared and used. However, linear alkylbenzenesulfonates do not have adequate property profiles in all areas of application.

First, for example, it would be advantageous to improve their low-temperature washing properties or their properties in hard water. Likewise desirable is the ready ability to be formulated, given by the viscosity of the sulfonates and their solubility. These improved properties are displayed by slightly branched compounds or mixtures of slightly branched compounds with linear compounds, although it is imperative to achieve the correct degree of branching and/or the correct degree of mixing. Too much branching adversely affects the biodegradability of the products. Products which are too linear have a negative effect on the viscosity and the solubility of the sulfonates.

Moreover, the ratio of terminal phenylalkanes (2-phenylalkanes and 3-phenylalkanes) relative to internal phenylalkanes (4-, 5-, 6- etc. phenylalkanes) plays a role for the product properties. A 2-phenyl fraction of about 20-40% and a 2- and 3-phenyl fraction of about 40-60% can be advantageous with regard to product quality (solubility, viscosity, washing properties, biodegradability).

Surfactants with very high 2- and 3-phenyl contents can have the considerable disadvantage that the processability of the products suffers as a result of a sharp increase in the viscosity of the sulfonates.

Moreover, the solubility behavior may not be optimum. Thus, for example, the Krafft point of a solution of LAS with very high or very low 2- or 3-phenyl fractions is up to 10-20° C. higher than in the case of the optimal choice of the 2- and 3-phenyl fraction.

BR 9204326 relates to the alkylation of aromatics with linear olefins over modified faujasite zeolites.

EP-A-0 160144 describes the alkylation of aromatics having predominantly long-chain olefins (e.g. C₁₆) over partially collapsed FAU structures.

U.S. Pat. No. 5,030,586 describes the drying of an aromatic and olefinic feedstock and the subsequent alkylation over FAU or BEA zeolites. Preference is given to using ethene and propene as olefinic feed substances.

U.S. Pat. No. 4,990,718 describes the di- and oligomerization of C₆₋₁₄-alpha-olefins and the subsequent alkylation of aromatic hydrocarbons with the dimerization products which have a branching ratio of 0.1-0.19, over zeolites having a pore size of 6.4-7.5 Å, predominantly zeolites of the faujasite type.

WO 99/05241 relates to cleaners which comprise branched alkylarylsulfonates as surfactants. The alkylarylsulfonates are obtained by dimerization of olefins to give vinylidine olefins, and subsequent alkylation of benzene over a shape-selective catalyst, such as MOR or BEA. This is followed by sulfonation.

WO 90/14160 describes specific zeolites of the faujasite type for the alkylation. Ethylbenzene and cumene are prepared using these catalysts.

The olefins used hitherto for the alkylation either have no branches at all, which contradicts the conception of the present invention, or some exhibit too high or too low a degree of branching, or produce a ratio of terminal to internal phenylalkanes which is not optimal. Others are prepared from expensive starting materials such as, for example, propene or alpha-olefins, and sometimes the proportion of the olefin fractions which is of interest for the preparation of surfactants is only about 20%. This leads to expensive work-up steps. Moreover, catalysts are used whose low space-time yields, high deactivation rates and high catalyst costs prevent an economic realization of the processes.

The object of the present invention is to provide a process for the preparation of alkylarylsulfonates or the alkylaryl compounds on which they are based, which are at least partially branched and thus have advantageous properties for use in detergents and cleaners compared with known compounds. In particular, they should have a suitable profile of properties of biodegradability, insensitivity toward water hardness, solubility and viscosity during the preparation and during use. In addition, the alkylarylsulfonates should be preparable in a cost-effective manner.

We have found that this object is achieved according to the invention by a process for the preparation of alkylaryl compounds by

1) preparation of a mixture of, on statistical average, predominantly monobranched C₁₀₋₁₄-olefins by

a) reaction of a C₄-olefin mixture over a metathesis catalyst for the preparation of an olefin mixture comprising 2-pentene and/or 3-hexene, and optional removal of 2-pentene and/or 3-hexene, followed by dimerization of the resulting 2-pentene and/or 3-hexene over a dimerization catalyst to give a mixture comprising C₁₀₋₁₂-olefins, and optionally removal of the C₁₀₋₁₂-olefins, or

b) extraction of predominantly monobranched paraffins from kerosene cuts and subsequent dehydrogenation, or

c) Fischer-Tropsch synthesis of olefins or paraffins, where the paraffins are dehydrogenated, or

d) dimerization of shorter-chain internal olefins, or

e) isomerization of linear olefins or paraffins, where the isomerized paraffins are dehydrogenated,

2) reaction of the olefin mixture obtained in stage 1) with an aromatic hydrocarbon in the presence of an alkylation catalyst which contains zeolites of the faujasite type.

The resulting alkylaryl compounds are subsequently sulfonated and neutralized in stage 3).

The combination of faujasite zeolite as alkylation catalyst with the olefins obtained from stages 1b) to 1e) gives products which, after sulfonation and neutralization, produce surfactants which have surprising properties, in particular with regard to sensitivity toward ions forming hardness, the solubility of the sulfonates, the viscosity of the sulfonates and their washing properties. Moreover, the present process is extremely cost-effective since the product streams can be arranged flexibly such that no by-products are formed.

Starting from a C₄ stream, in stage 1a), the metathesis produces linear, internal olefins which are then converted into branched olefins via the dimerization step.

The process according to the invention, with stage 1b, offers the essential advantage that the combination of metathesis and dimerization produces an olefin mixture which, following alkylation of an aromatic with the catalysts according to the invention, sulfonation and neutralization, produces a surfactant which is notable for its combination of excellent application properties (solubility, viscosity, stability to water hardness, washing properties, biodegradability). With regard to the biodegradability of alkylarylsulfonates, compounds which are less strongly adsorbed to sewage sludge or, as a result of reduced precipitation by water hardness, have higher bioavailability than conventional LAS are particularly advantageous.

According to the invention, the processes for the preparation of alkylarylsulfonates can have the following features:

Preparation of a mixture of slightly branched olefins having an overall carbon number of 10-14.

Reaction of the olefin mixture obtained in stage 1) with an aromatic hydrocarbon in the presence of an alkylation catalyst of the faujasite type to form alkylaromatic compounds, it being possible to mix in additional linear olefins before the reaction.

Sulfonation and neutralization of the alkylaromatic compounds obtained in stage 2) and neutralization to alkylarylsulfonates, it being possible to additionally add linear alkylbenzenes prior to the sulfonation.

Optionally mixing of the alkylarylsulfonates obtained in stage 2) with linear alkylarylsulfonates.

Stage 1) of the process according to the invention is the preparation of a mixture of slightly branched olefins having an overall carbon number of 10-14.

1a)

Preference is given to the reaction of a C₄-olefin mixture over a metathesis catalyst for the preparation of an olefin mixture comprising 2-pentene and/or 3-hexene, and optionally removal of 2-pentene and/or 3-hexene. The metathesis can be carried out, for example, as described in DE-A-199 32 060. The resulting 2-pentene and/or 3-hexene is dimerized over a dimerization catalyst to give a C₁₀₋₁₂-olefin mixture. The C₁₀₋₁₂-olefins obtained are optionally separated off.

The metathesis reaction is here preferably carried out in the presence of heterogeneous metathesis catalysts which are not or only slightly isomerization-active and are selected from the class of transition metal compounds of metals of group VIb, VIIb or VIII of the Periodic Table of the Elements applied to inorganic supports.

The preferred metathesis catalyst used is rhenium oxide on a support, preferably on γ-aluminum oxide or on Al₂O₃/B₂O₃/SiO₂ mixed supports.

In particular, the catalyst used is Re₂O₇/γ-Al₂O₃ with a rhenium oxide content of from 1 to 20% by weight, preferably 3 to 15% by weight, particularly preferably 6 to 12% by weight.

The metathesis is, when carried out in a liquid phase, preferably carried out at a temperature of from 0 to 150° C., particularly preferably 20-80° C., and at a pressure of 2-200 bar, particularly preferably 5-30 bar.

If the metathesis is carried out in the gas phase, the temperature is preferably 20 to 300° C., particularly preferably 50 to 200° C. The pressure in this case is preferably 1 to 20 bar, particularly preferably 1 to 5 bar.

The preparation of C₅/C₆-olefins and optionally propene from steam cracker or refinery C₄ streams may comprise the substeps (1) to (4):

(1) removal of butadiene and acetylenic compounds by optional extraction of butadiene with a butadiene-selective solvent and subsequently /or selective hydrogenation of butadienes and acetylenic impurities present in crude C₄ fraction to give a reaction product which comprises n-butenes and isobutene and essentially no butadienes and acetylenic compounds,

(2) removal of isobutene by reaction of the reaction product obtained in the previous stage with an alcohol in the presence of an acidic catalyst to give an ether, removal of the ether and the alcohol, which can be carried out simultaneously with or after the etherification, to give a reaction product which comprises n-butenes and optionally oxygen-containing impurities, it being possible to discharge the ether formed or back-cleave it to obtain pure isobutene, and to follow the etherification step by a distillation step for the removal of isobutene, where, optionally, introduced C₃-, i-C₄- and C₅-hydrocarbons can also be removed by distillation during the work-up of the ether, or oligomerization or polymerization of isobutene from the reaction product obtained in the previous stage in the presence of an acidic catalyst whose acid strength is suitable for the selective removal of isobutene as oligoisobutene or polyisobutene, to give a stream containing 0 to 15% of residual isobutene,

(3) removal of the oxygen-containing impurities from the product of the preceding steps over appropriately selected adsorber materials,

(4) metathesis reaction of the resulting raffinate II stream as described.

The substep of selective hydrogenation of butadiene and acetylenic impurities present in crude C₄ fraction is preferably carried out in two stages by bringing the crude C₄ fraction in the liquid phase into contact with a catalyst which comprises at least one metal selected from the group consisting of nickel, palladium and platinum on a support, preferably palladium on aluminum oxide, at a temperature of from 20 to 200° C., a pressure of from 1 to 50 bar, a volume flow rate of from 0.5 to 30 m³ of fresh feed per m³ of catalyst per hour and a ratio of recycle to feed stream of from 0 to 30 with a molar ratio of hydrogen to diolefins of from 0.5 to 50, to give a reaction product in which, apart from isobutene, the n-butenes 1-butene and 2-butene are present in a molar ratio of from 2:1 to 1:10, preferably from 2:1 to 1:3, and essentially no diolefins and acetylenic compounds are present. For a maximum yield of hexene, 1-butene is preferably present in excess, and for a high protein yield, 2-butene is preferably present in excess. This means that the overall molar ratio in the first case canbe 2:1 to 1:1 and in the second case 1:1 to 1:3.

The substep of butadiene extraction from crude C₄ fraction is preferably carried out using a butadiene-selective solvent selected from the class of polar-aprotic solvents, such as acetone, furfural, acetonitrile, dimethylacetamide, dimethylformamide and N-methylpyrrolidone, to give a reaction product in which, following subsequent selective hydrogenation/isomerization, the n-butenes 1-butene and 2-butene are present in a molar ratio 2:1 to 1:01, preferably from 2:1 to 1:3.

The substep of isobutene etherification is preferably carried out in a three-stage reactor cascade using methanol or isobutanol, preferably isobutanol, in the presence of an acidic ion exchanger, in which the stream to be etherified flows downwardly through flooded fixed-bed catalysts, the rector inlet temperature being 0 to 60° C., preferably 10 to 50° C., the outlet temperature being 25 to 85° C., preferably 35 to 75° C., the pressure being 2 to 50 bar, preferably 3 to 20 bar, and the ratio of isobutanol to isobutene being 0.8 to 2.0, preferably 1.0 to 1.5, and the overall conversion corresponding to the equilibrium conversion.

The substep of isobutene removal is preferably carried out by oligomerization or polymerization of isobutene starting from the reaction mixture obtained after the above-described stages of butadiene extraction and/or selective hydrogenation, in the presence of a catalyst selected from the class of homogeneous and heterogeneous Broensted or Lewis acids, see DE-A-100 13 253.

Dimerization of the olefins or olefin mixtures present in the metathesis step gives dimerization products which, with regard to further processing to alkylaromatics, have particularly favorable components and particularly advantageous compositions.

For a more detailed description of the metathesis/dimerization process and the upstream steps, reference is made to DE-A-199 32 060.

In addition to the metathesis/dimerization reaction described above, it is, however, also possible to carry out conventional processes for the preparation of slightly branched olefins. This is e.g. 1b) the extraction of i-paraffins from diesel/kerosene fractions which are formed either in the processing and refining of crude oil, or 1c) are formed by synthetic processes such as, for example, the Fischer-Tropsch synthesis, and optionally subsequent dehydrogenation of the i-paraffins to i-olefins.

Moreover, slightly branched olefins can be prepared e.g. 1d) by the dimerization of shorter-chain olefins.

A further possibility represents, for example, 1e) the isomerization of suitable linear olefins to slightly branched olefins.

Stage 2) is the reaction of the olefin mixture obtained in stage 1) with an aromatic hydrocarbon in the presence of an alkylation catalyst of the faujasite type to form alkylaromatic compounds, it being possible to mix in additional linear olefins prior to the reaction.

Here, preference is given to using an alkylation catalyst which leads to alkylaromatic compounds which, in the alkyl radical, have 1 to 3 carbon atoms with an H/C index of 1, or the reaction conditions are chosen accordingly.

In choosing the faujasite catalyst used according to the invention, attention must be paid, regardless of the great effect of the feedstock used, to the minimizing of compounds formed by the catalyst which are characterized in that they include carbon atoms with an H/C index of 0 in the side chain. The proportion of carbon atoms in the alkyl radical with an H/C index of 0 should, on statistical average of all compounds, be less than 5% (preferably less than 1%).

The H/C index defines the number of protons per carbon atom.

The olefins used according to the process of the invention preferably have no carbon atoms with an H/C index of 0 in the side chain. If, then, the alkylation of the aromatic is carried out using the olefin under conditions as described here and under which no skeletal isomerization of olefin takes place, then carbon atoms with an H/C index of 0 may form only in the benzyl position relative to the aromatic, i.e. it suffices to determine the H/C index of the benzylic carbon atoms.

Furthermore, the intention is to form compounds which, on average, have 1 to 3 carbon atoms with an H/C index of 1 in the side chain. This is achieved, in particular, by the choice of a suitable feedstock and also suitable catalysts which, on the one hand, as a result of their geometry, suppress the formation of undesired products, but, on the other hand, permit an adequate reaction rate.

Catalysts for the process according to the invention are zeolites of the faujasite type, in particular zeolite Y and modifications thereof. Modifications is understood as meaning modified faujasites which may be prepared, for example, by processes such as ion exchange, steaming, blocking of external centers, etc. The catalysts are characterized in particular by the fact that, in the X-ray powder diffractogram, they contain more than 20% of a phase which can be indicated with the cubic structure of the faujasite.

Although in the published literature (e.g. Cao et al., Appl. Catal. 184 (1999) 231; Sivasanker et al., J. Catal. 138 (1992) 386; Liang et al., Zeolites 17 (1996) 297; Almeida et al., Appl. Catal. 114 (1994) 141) it has been shown that zeolites of the faujasite type (FAU) have, in contrast to the zeolites mordenite (MOR) and beta (BEA), virtually no shape selectivity in the alkylation of aromatics with linear olefins—a similar approach is to be found e.g. in WO 99/05082, where MOR and BEA zeolites are described for the reaction with branched olefins—it has, surprisingly, now been found that zeolites of the faujasite type exhibit shape-selective behavior in the alkylation of aromatic hydrocarbons (preferably benzene) with slightly branched olefins (preferably those from a metathesis/dimerization stage 1b)) and, moreover, produce an optimum proportion of 2- and 3-phenylalkanes, coupled with simultaneously low catalyst costs—for example, HY is currently about 3-4 times less expensive than H-MOR or H-BEA, have economically interesting space/time yields and a moderate deactivation behavior.

In heterogeneous catalysis, shape selectivity describes the phenomenon of excluding starting materials, transition states or products from participating in the reaction, or not permitting them in the reaction as a result of a steric hindrance prescribed by the catalyst. With regard to the alkylbenzenes and alkylbenzenesulfonates according to the invention, in particular with regard to their H/C indices, this phenomenon is of decisive importance. While with non-shape-selective catalysts products are obtained which include carbon atoms with H/C indices of 0 in the side chain, these compounds are excluded according to the invention using shape-selective catalysts.

Catalysts with narrow pore systems, however, always have the disadvantage that the achievable space/time yields turn out to be lower than in the case of catalysts with larger pores or in the case of macro- or mesoporous substances. For this reason, it is important to find a catalyst which both satisfies the precondition of the correspondingly desired shape selectivity, but additionally also has the highest possible space/time yields, such that nothing stands in the way of an economic realization of the process.

Moreover, it is known that pore systems which are too narrow are subject to severe and rapid deactivation, which likewise impairs the efficiency of the process as a result of the need for frequent regenerations of the catalysts.

Moreover, in choosing the catalysts, their tendency with regard to deactivation should be taken into consideration. One-dimensional pore systems in most cases have the disadvantage of rapid blocking of the pores as a result of degradation or formative products from the process. Moreover, the inhibition of diffusion of the reactants and the products in one-dimensional pore systems is greater than in polydimensional pore systems. Catalysts with polydimensional pore systems are therefore to be preferred.

The catalysts used may be of natural or synthetic origin, the properties of which can be adjusted to a certain extent by methods known from the literature, as are described, for example, in J. Weitkamp and L. Puppe, Catalysis and Zeolites, Fundamentals and Applications, chapter 3: G. Kühl, Modification of Zeolites, Springer Verlag, Berlin, 1999 (ion exchange, dealuminization, dehydroxylation and extraction of lattice aluminum, thermal treatment, steaming, treatment with acids or SiCl₄, blocking of specific, e.g. external, azidic centers by e.g. silylation, reinsertion of aluminum, treatment with aluminum halides and oxo acids). It is important for the present invention that the catalysts have more than 10 □mol/g of acidic centers at a pKa value of less than 3.3. The number of acidic centers is determined here in accordance with the Hammett titration method using dimethyl yellow [CAS No. 60-11-7] as indicator and n-butylamine as probe in accordance with H.A. Benesi and B.H.C. Winquist in Adv. Catal., vol. 27, Academic Press 1978, p. 100 ff.

Furthermore, the catalysts can also contain already spent catalyst material or consist of material which has been regenerated by customary methods, e.g. by a recalcination in air, H₂O, CO₂ or inert gase at temperatures greater than 200° C., by washing with H₂O, acids or organic solvents, by steaming or by treatment under reduced pressure at temperatures greater than 200° C.

They can be used in the form of powders or, preferably, in the form of moldings, such as extrudates, tablets or chips. For the shaping 2 to 60% by weight (based on the mass to be shaped) of binders may be added. Suitable binders are various aluminum oxides, preferably boehmite, amorphous aluminosilicates having a molar SiO₂/Al₂O₃ ratio of 25:75 to 95:5, silicon dioxide, preferably highly disperse SiO₂, such as e.g. silica sols, mixtures of highly disperse SiO₂ and highly disperse Al₂O₃, highly disperse TiO₂, and clays. Following shaping, the extrudates or compacts are advantageously dried at 110° C./16 h and calcined at 300 to 500° C. for 2 to 16 h, it also being possible to carry out the calcination directly in the alkylation reactor.

As a rule, the catalysts are used in the H form. To increase the selectivity, the service life and the number of possible catalyst regenerations, it is, however, possible to undertake various modifications on the catalysts in addition.

A modification of the catalysts consists in exchanging or doping the unshaped catalysts with alkali metals, such as Na and K, alkaline earth metals, such as Ca, Mg, earth metals, such as Tl, transition metals, such as, for example, Mn, Fe, Mo, Cu, Zn, Cr, precious metals and/or rare earth metals, such as, for example, La, Ce or Y ions.

An advantageous catalyst embodiment consists in placing the shaped catalysts in a flow tube and, at 20 to 100° C., passing over, for example, a halide, an acetate, an oxalate, a citrate or a nitrate of the above-described metals in dissolved form. Ion exchange of this type can be carried out, for example, on the hydrogen, ammonium or alkali metal form of the catalysts.

Another way of applying the metal to the catalysts consists in impregnating the zeolitic material with, for example, a halide, acetate, oxalate, citrate, nitrate or oxide of the above-described metals in aqueous or alcoholic solution.

Both ion exchange and also impregnation can be followed by drying, or alternatively repeated calcination. In the case of metal-doped catalysts, an aftertreatment with hydrogen and/or with steam may be favorable.

A further possibility of modifying the catalyst consists in subjecting the heterogeneouscatalytic material, in shaped or unshaped form, to treatment with acids, such as hydrochloric acid (HCl), hydrofluoric acid (HF), phosphoric acid (H₃PO₄), sulfuric acid (H₂SO₄), oxalic acid (HO₂C-CO₂H) or mixtures thereof.

A particular embodiment consists in treating the catalyst powder prior to its shaping with hydrofluoric acid (0.001 to 2 molar, preferably 0.05 to 0.5 molar) for 1 to 3 hours with reflux. After the product has been filtered off and washed, it is usually dried at 100 to 160° C. and calcined at 400 to 550° C.

A further particular embodiment consists in an HCl treatment of the heterogeneous catalysts following their shaping with binders. Here, the heterogeneous catalyst is usually treated for 1 to 3 hours at temperatures between 60 and 80° C. with a 3 to 25% strength, in particular with a 12 to 20% strength, hydrochloric acid, then washed, dried at 100 to 160° C. and calcined at 400 to 550° C.

Another possible modification of the catalyst is the exchange with ammonium salts, e.g. with NH₄Cl, or with mono-, di- or polyamines. For this, the heterogeneous catalyst shaped with binders is subjected to exchange with from 10 to 25% strength, preferably about 20% strength, NH₄Cl solution, usually at 60 to 80° C., continuously for 2 h in heterogeneous catalyst/ammonium chloride solution in a weight ratio of 1:15, and then dried at 100 to 120° C.

A further modification which can be carried out on aluminum-containing catalysts is dealuminization, where some of the aluminum atoms are replaced by silicon or the aluminum content of the catalysts is decreased by, for example, hydrothermal treatment.

Hydrothermal dealuminization is advantageously followed by extraction with acids or complexing agents in order to remove non-lattice aluminum formed. The replacement of aluminum by silicon can be carried out, for example, using (NH₄)₂SiF₆ or SiCl₄. Examples of dealuminizations of Y zeolites are given in Corma et al., Stud. Surf. Sci. Catal. 37 (1987), pages 495 to 503.

The modification by silylation is described in general terms in J. Weitkamp and L. Puppe, Catalysis and Zeolites, Fundamentals and Applications, chapter 3: G. Kühl, Modification of Zeolites, Springer Verlag, Berlin, 1999. The procedure usually involves selectively blocking azidic centers, e.g. external ones by bulky bases such as, for example, 2,2,6,6-tetramethylpiperidine or 2,6-lutidine, and then treating the zeolite with suitable Si compounds, such as, for example, tetraethyl orthosilicate, tetramethyl orthosilicate, C1-C20-trialkylsilyl chloride, methoxide or ethoxide or SiCl₄. This treatment can be carried out either with gaseous Si compounds or with Si compounds dissolved in anhydrous solvents, such as, for example, hydrocarbons or alcohols. A combination of different Si compounds is also possible. Alternatively, the Si compound can also already contain the amine group selective for azidic centers, such as, for example, 2,6-trimethylsilylpiperidine. The catalysts modified in this way are then usually calcined at temperatures of from 200 to 500° C. in O₂-containing atmosphere.

A further modification consists in the blockading of external centers by mixing or grinding the catalyst powder with metal oxides, such as, for example, MgO, and subsequent calcination at 200-500° C.

The catalysts can be used for the alkylation of aromatics as extrudates having diameters of e.g. 1 to 4 mm or as tablets having diameters of e.g. 3 to 5 mm.

The type of aliphatic raw material used according to the invention, and the choice of catalyst according to the invention lead to the ratios, optimal for detergent and cleaning applications, of 2-, 3-, 4-, 5- and 6-phenylalkanes. Preference is given to the preparation of a 2-phenyl fraction of 20-40% and a 2- and 3-phenyl fraction of 40-60%.

### Preferred Reaction Method

The alkylation is carried out by allowing the aromatic compounds (the aromatic compound mixture) and the olefin (mixture) to react in a suitable reaction zone by bringing them into contact with the catalyst, working up the reaction mixture after the reaction and thus obtaining the desired products.

Suitable reaction zones are, for example, tubular reactors, stirred-tank reactors or a stirred-tank reactor battery, a fluidized bed, a loop reactor or a solid/liquid moving bed. When the catalyst is in solid form, then it can be used either as a slurry, as a fixed bed, as a moving bed or as a fluidized bed.

Where a fixed-bed reactor is used, the reactants can be introduced either in cocurrent or in countercurrent. Realization as a catalytic distillation is also possible.

The reactants are either in the liquid and/or in the gaseous state, but preferably in the liquid state. The reaction is also possible in the supercritical state.

The reaction temperature is chosen such that, on the one hand, as complete as possible a conversion of the olefin takes place and, on the other hand, the fewest possible by-products arise. By-products are, in particular, dialkylbenzenes, diphenylalkanes and olefin oligomers. The choice of temperature also depends decisively on the catalyst chosen. Reaction temperatures between 50° C. and 500° C. (preferably 80 to 350° C., particularly preferably 80-250° C.) can also be used.

The pressure of the reaction depends on the procedure chosen (reactor type) and is between 0.1 and 100 bar, and the WHSV is chosen between 0.1 and 100.

The reactants can optionally be diluted with inert substances. Inert substances are preferably paraffins.

The molar ratio of aromatic compound:olefin is usually set between 1:1 and 100:1 (preferably 2:1-20:1).

The process can be carried out discontinuously, semicontinuously by initially introducing, for example, catalyst and aromatic, and metering in olefin, or fully continuously, optionally also with the continuous feed and discharge of catalyst.

Catalyst with insufficient activity can be regenerated directly in the alkylation reactor or in a separate unit by

1) washing with solvents, such as, for example, alkanes, aromatics, such as, for example, benzene, toluene or xylene, ethers, such as, for example, tetrahydrofuran, tetrahydropyran, dioxane, dioxolane, diethyl ether or methyl t-butyl ether, alcohols, such as, for example, methanol, ethanol, propanol and isopropanol, amides, such as, for example, dimethylformamide or formamide, nitriles, such as, for example, acrylonitrile or water, at temperatures of from 20 to 200° C.,

2) by treatment with water vapor at temperatures of from 100° C. to 400° C.

3) by thermal treatment in reactive gas atmosphere (O₂ and O₂-containing gas mixtures, such as CO₂, CO, H₂) at 200-600° C. or

4) by thermal treatment in an inert gas atmosphere (N₂, noble gases) at 200-600° C. Alternatively, deactivated catalyst can, as described above, also be added during the preparation of new catalyst.

### Aromatic Feed Substances

All aromatic hydrocarbons of the formula Ar-R are possible, where Ar is a monocyclic or bicyclic aromatic hydrocarbon radical, and R is chosen from H, C₁₋₅, preferably C₁₋₃-alkyl, OH, OR etc., preferably H or C₁₋₃-alkyl. Preference is given to benzene and toluene.

Stage 3)

In stage 3), the alkylaromatic compounds obtained in stage 2) are sulfonated and neutralized to give alkylarylsulfonates. Alkylaryls are converted into alkylarylsulfonates by

sulfonation (e.g. with SO₃, oleum, chlorosulfonic acid, etc., preferably with SO₃) and subsequent

neutralization (e.g. with Na, K, NH₄, Mg compounds, preferably with Na compounds).

Sulfonation and neutralization are adequately described in the literature and are carried out in accordance with the prior art. The sulfonation is preferably carried out in a falling-film reactor, but can also be carried out in a stirred-tank reactor. The sulfonation with SO₃ is to be preferred over the sulfonation with oleum.

### Mixtures

The compounds prepared by processes described above are further processed (preferably) either as such, or are mixed beforehand with other alkylaryls and then passed to the further processing step. In order to simplify this process, it may also be sensible to mix the raw materials which are used for the preparation of the other alkylaryls mentioned above directly with the raw materials of the present process, and then to carry out the process according to the invention. Thus, the mixing of slightly branched olefin streams from the process according to the invention with linear olefins, for example, is sensible. Mixtures of the alkylaryl-sulfonic acids or of the alkylarylsulfonates can also be used. The mixings are always undertaken with regard to optimization of the product quality of the surfactants prepared from the alkylaryl.

An exemplary overview of alkylation, sulfonation, neutralization is given, for example, in “Alkylaryl-sulfonates: History, Manufacture, Analysis and Environmental Properties” in Surf. Sci. Ser. 56 (1996) Chapter 2, Marcel Dekker, New York, and references contained therein.

### Analysis of the Structural Parameters

During the alkylation of aromatics with olefins, alkylaromatics of the formulae R′″ArCH₂R (1), R′″ArCHRR′ (2) and R′″ArCRR′R″ (3) arise. R′″is H or C₁-C₃-alkyl. The proportions of (1)-(3) are determined as shown below using the example of benzene as aromatic:

1) The reactor discharge is distilled and unreacted aromatic, unreacted olefin and heavy alkylate formed by alkylation of the aromatic with more than one molecule of olefin are separated off.

2) The proportion of (1) is then determined as follows:

25 mg of alkylbenzene and 5 mg of chromium acetylacetonate (CAS 21679-31-2) are dissolved in 500 mg of CDCl3 and transferred to an NMR sample tube with an internal diameter of 5 mm. Then, with an inverse gated pulse sequence every 6 s, a C13 NMR spectrum is recorded at a measurement frequency of 125 MHz, and 6 000 of these spectra are determined. The sum spectrum is then normalized to CDCl₃=77.47 ppm. The proportion of structures of type (1) is then given by

proportion of (1)=(integral from 139 to 143.5 ppm)/(integral from 139 to 152 ppm)

3) The proportion of (2) is then determined as follows:

5 mg of alkylbenzene and 0.5 mg of SiMe₄ are dissolved in 500 mg of CDCl₃ and transferred to an NMR sample tube with an internal diameter of 5 mm. Then, with a 30° pulse sequence every 5 s, an H1 NMR spectrum is recorded at a measurement frequency of 500 MHz, and 32 of these spectra are determined. The sum spectrum is then normalized to SiMe₄=0 ppm. The proportion of structures of the type (2) is then given by

proportion of (2)=5* (integral from 2.2 to 3.2 ppm)/(integral from 6.9 to 7.6 ppm)−2* proportion of (1)

3) The proportion of (3) is then given by the normalization condition

proportion of (1)+proportion of (2)+proportion of (3)=100%.

The determination of aromatics different from benzene is carried out analogously.

The invention also relates to alkylaryl compounds and alkylarylsulfonates obtainable by a process as described above.

The alkylarylsulfonates according to the invention are preferably used as surfactants, in particular in detergents and cleaners. The invention also relates to detergents and cleaners comprising, in addition to customary ingredients, alkylarylsulfonates as described above.

Nonexhaustive examples of customary ingredients of detergents and cleaners according to the invention are listed below.

### Bleach

Examples are alkali metal perborates or alkali metal carbonate perhydrates, in particular the sodium salts.

One example of an organic peracid which can be used is peracetic acid, which is preferably used in commercial textile washing or commercial cleaning.

Bleach or textile detergent compositions which can be used advantageously comprise C₁-₁₂-percarboxylic acids, C₈₋₁₆-dipercarboxylic acids, imidopercarboxylic acids or aryldipercarboxylic acids. Preferred examples of acids which can be used are peracetic acid, linear or branched octane-, nonane-, decane- or dodecane-monoper-acids, decane- and dodecane-diperacid, mono- and diperphthalic acids, -isophthalic acids and -terephthalic acids, phthalimidopercaproic acid and terephthaloyldipercaproic acid. It is likewise possible to use polymeric peracids, for example those which contain the acrylic acid basic building blocks in which a peroxy function is present. The percarboxylic acids may be used as free acids or as salts of the acids, preferably alkali metal or alkaline earth metal salts.

### Bleach Activator

Bleach catalysts are, for example, quatemized imines and sulfonimines, as described, for example, in U.S. Pat. No. 5,360,568, U.S. Pat. No. 5,360,569 and EP-A-0 453 003, and also manganese complexes as described, for example, in WO-A 94/21777. Further metal-containing bleach catalysts which may be used are described in EP-A-0 458 397, EP-A-0 458 398, EP-A-0 549 272.

Bleach activators are, for example, compounds from the classes of substance below: polyacylated sugars or sugar derivatives having C₁₋₁₀-acyl radicals, preferably acetyl, propionyl, octanoyl, nonanoyl or benzoyl radicals, particularly preferably acetyl radicals, can be used as bleach activators. As sugars or sugar derivatives, it is possible to use monoor disaccharides, and reduced or oxidized derivatives thereof, preferably glucose, mannose, fructose, sucrose, xylose of lactose. Particularly suitable bleach activators of this class of substance are, for example, pentacetylglucose, xylose tetraacetate, 1-benzoyl-2,3,4,6-tetraacetylglucose and 1-octanoyl-2,3,4,6-tetraacetylglucose.

A further class of substance which can be used comprises the acyloxybenzenesulfonic acids and alkali metal and alkaline earth metal salts thereof, it being possible to use C-₁₋₁₄-acyl radicals. Preference is given to acetyl, propionyl, octanoyl, nonanoyl and benzoyl radicals, in particular acetyl radicals and nonanoyl radicals. Particularly suitable bleach activators from this class of substance are acetyloxybenzenesulfonic acid. They are preferably used in the form of their sodium salts.

It is also possible to use O-acyl oxime esters, such as, for example, O-acetylacetone oxime, O-benzoyl-acetone oxime, bis(propylamino) carbonate, bis(cyclo-hexylimino) carbonate. Examples of acylated oximes which can be used according to the invention are described, for example, in EP-A-0 028 432. Oxime esters which can be used according to the invention are described, for example, EP-A-0 267 046.

It is likewise possible to use N-acylcaprolactams, such as, for example, N-acetylcaprolactam, N-benzoylcapro-lactam, N-octanoylcaprolactam, carbonylbiscaprolactam.

It is also possible to use

N-diacylated and N,N′-tetraacylated amines, e.g. N,N,N′,N′-tetraacetylmethylenediamine and -ethylenediamine (TADE), N,N-diacetylaniline, N,N-diacetyl-p-toluidine or 1,3-diacylated hydantoins, such as 1,3-diactyl-5,5-dimethyl-hydantoin;

N-alkyl-N-sulfonylcarboxamides, e.g. N-methyl-N-mesylacetamide or N-methyl-N-mesylbenzamide;

N-acylated cyclic hydrazides, acylated triazoles or urazoles, e.g. monoacetylmaleic hydrazide;

O,N,N-trisubstituted hydroxylamines, e.g. O-benzoyl-N,N-succinylhydroxylamine, O-acetyl-N,N-succinylhydroxylamine or O,N,N-triacetylhydroxyl-amine;

N,N′-diacylsulfurylamides, e.g. N,N′-dimethyl-N,N′-diacetylsulfurylamide or N,N′-diethyl-N,N′-di-propionylsulfurylamide;

triacyl cyanurate, e.g. triacetyl cyanurate or tribenzoyl cyanurate;

carboxylic anhydrides, e.g. benzoic anhydride, m-chlorobenzoic anhydride or phthalic anhydride;

1,3-diacyl-4,5-diacyloxyimidazolines, e.g. 1,3-diacetyl-4,5-diacetoxyimidazoline;

tetraacetylglycoluril and tetrapropionylglycoluril;

diacylated 2,5-diketopiperazines, e.g. 1,4-diacetyl-2,5-diketopiperazine;

acylation products of propylenediurea and 2,2,-di-methylpropylenediurea, e.g. tetraacetylpropylene-diurea;

α-acyloxypolyacylmalonamides, e.g. a-acetoxy-N,N′-diacetylmalonamide;

diacyldioxohexahydro-1,3,5-triazines, e.g. 1,5-diacetyl-2,4-dioxohexahydro-1,3,5-triazine.

It is likewise possible to use 1-alkyl- or 1-aryl-(4H)-3,1-benzoxazin-4-ones, as are described, for example, in EP-B1-0 332 294 and EP-B 0 502 013. In particular, it is possible to use 2-phenyl-(4H)-3,1-benzoxazin-4-one and 2-methyl-(4H)-3,1-benzoxazin-4-one.

It is also possible to use cationic nitriles, as described, for example, in EP 303 520 and EP 458 391 A1. Examples of suitable cationic nitrites are the methosulfates or tosylates of trimethylammoniumacetonitrile, N,N-dimethyl-N-octyl-ammoniumacetonitrile, 2-(trimethylammonium)propio-nitrile, 2-(trimethylammonium)-2-methylpropionitrile, N-methylpiperazinium-N,N′-diacetonitrile and N-methyl-morpholiniumacetonitrile.

Particularly suitable crystalline bleach activators are tetraacetylethylenediamine (TAED), NOBS, isoNOBS, carbonylbiscaprolactam, benzoylcaprolactam, bis(2-propylimino) carbonate, bis(cyclohexylimino) carbonate, O-benzoylacetone oxime and 1-phenyl-(4H)-3,1-benzoxazin-4-one, anthranil, phenylanthranil, N-methylmorpholinoacetonitrile, N-octanoylcaprolactam (OCL) and N-methylpiperazine-N,N′-diacetonitrile, and liquid or poorly crystallizing bleach activators in a form formulated as a solid product.

### Bleach Stabilizer

This comprises additives which are able to adsorb, bind or complex traces of heavy metal. Examples of additives with a bleach-stabilizing action which can be used according to the invention are polyanionic compounds, such as polyphosphates, polycarboxylates, polyhydroxy-polycarboxylates, soluble silicates in the form of completely or partially neutralized alkali metal or alkaline earth metal salts, in particular in the form of neutral Na or Mg salts, which are relatively weak bleach stabilizers. Strong bleach stabilizers which can be used according to the invention are, for example, complexing agents, such as ethylenediaminetetraacetate (EDTA), nitrilotriacetic acid (NTA), methylglycine-diacetic acid (MGDA), β-alaninediacetic acid (ADA), ethylenediamine-N,N′-disuccinate (EDDS) and phosphonates, such as ethylenediaminetetramethylene-phosphonate, diethylenetriaminepentamethylene-phosphonate or hydroxyethylidene-1,1-diphosphonic acid in the form of the acids or as partially or completely neutralized alkali metal salts. The complexing agents are preferably used in the form of their Na salts.

In the field of textile washing, bleaching and household cleaning and in the commercial sector, the bleach or textile detergent compositions described may, in accordance with one embodiment of the invention, comprise virtually all customary constituents of detergents, bleaches and cleaners. In this way, it is possible, for example, to formulate compositions which are specifically suitable for textile treatment at low temperatures, and also those which are suitable in a number of temperature ranges up to and including the traditional range of the boil wash.

In addition to bleach compositions, the main constituents of textile detergents and cleaners are builders, i.e. inorganic builders and/or organic cobuilders, and surfactants, in particular anionic and/or nonionic surfactants. In addition, it is also possible for other customary auxiliaries and adjuncts, such as extenders, complexing agents, phosphonates, dyes, corrosion inhibitors, antiredeposition agents and/or soil release polymers, color-transfer inhibitors, bleach catalysts, peroxide stabilizers, electrolytes, optical brighteners, enzymes, perfume oils, foam regulators and activating substances, to be present in these compositions if this is advantageous.

### Inorganic Builders (Builder Substances)

Suitable inorganic builder substances are all customary inorganic builders, such as aluminosilicates, silicates, carbonates and phosphates.

Examples of suitable inorganic builders are alumino-silicates having ion-exchanging properties, such as, for example, zeolites. Various types of zeolites are suitable, in particular zeolite A, X, B, P, MAP and HS in their Na form or in forms in which Na has partially been replaced by other cations such Li, K, Ca, Mg or ammonium. Suitable zeolites are described, for example, in EP-A 038 591, EP-A 021 491, EP-A 087 035, U.S. Pat. No. 4,604,224, GB-A2 013 259, EP-A 522 726, EP-A 384 070 and WO-A 94/24 251.

Further suitable inorganic builders are, for example, amorphous or crystalline silicates, such as, for example, amorphous disilicates, crystalline disilicates, such as the phyllosilicate SKS-6 (manufacturer: Hoechst). The silicates can be used in the form of their alkali metal, alkaline earth metal or ammonium salts. Preference is given to using Na, Li and Mg silicates.

### Anionic Surfactants

Suitable anionic surfactants are the linear and/or slightly branched alkylbenzenesulfonates (LAS) according to the invention.

Further suitable anionic surfactants are, for example, fatty alcohol sulfates of fatty alcohols having 8 to 22, preferably 10 to 18, carbon atoms, e.g. C₉-C₁₁-alcohol sulfates, C₁₂-C₁₃-alcohol sulfates, cetyl sulfate, myristyl sulfate, palmityl sulfate, stearyl sulfate and tallow fatty alcohol sulfate.

Further suitable anionic surfactants are sulfated ethoxylated C₈-C₂₂-alcohols (alkyl ether sulfates) or soluble salts thereof Compounds of this type are prepared, for example, by firstly alkoxylating a C₈-C₂₂-alcohol, preferably a C₁₀-C₂₂-alcohol, e.g. a fatty alcohol, and then sulfating the alkoxylation product. For the alkoxylation, preference is given to using ethylene oxide, in which case 2 to 50 mol, preferably 3 to 20 mol, of ethylene oxide are used per mole of fatty alcohol. The alkoxylation of the alcohols can, however, also be carried out using propylene oxide on its own and optionally butylene oxide. Also suitable are those alkoxylated C₈-C₂₂-alcohols which contain ethylene oxide and propylene oxide or ethylene oxide and butylene oxide. The alkoxylated C₈-C₂₂-alcohols may contain the ethylene oxide, propylene oxide and butylene oxide units in the form of blocks or in random distribution.

Further suitable anionic surfactants are N-acylsarcosinates having aliphatic saturated or unsaturated C₈-C₂₅-acyl radicals, preferably C₁₀-C₂₀-acyl radicals, e.g. N-oleoylsarcosinate.

The anionic surfactants are preferably added to the detergent in the form of salts. Suitable cations in these salts are alkali metal salts, such as sodium, potassium and lithium and ammonium salts such as, for example, hydroxyethylammonium, di(hydroxyethyl)ammonium and tri(hydroxyethyl)ammonium salts.

The detergents according to the invention preferably comprise linear and/or slightly branched C₁₀-C₁₃-alkylbenzenesulfonates (LAS).

### Nonionic Surfactants

Suitable nonionic surfactants are, for example, alkoxylated C₈-C₂₂-alcohols, such as fatty alcohol alkoxylates or oxo alcohol alkoxylates. The alkoxylation can be carried out with ethylene oxide, propylene oxide and/or butylene oxide. Surfactants which can be used here are any alkoxylated alcohols which contain at least two molecules of an abovementioned alkylene oxide in added form. Block polymers of ethylene oxide, propylene oxide and/or butylene oxide are also suitable here, or addition products which contain said alkylene oxides in random distribution. Per mole of alcohol, 2 to 50 mol, preferably 3 to 20 mol, of at least one alkylene oxide are used. The alkylene oxide used is preferably ethylene oxide. The alcohols preferably have 10 to 18 carbon atoms.

A further class of suitable nonionic surfactants are alkylphenol ethoxylates having C₆-C₁₄-alkyl chains and 5 to 30 mol of ethylene oxide units.

Another class of nonionic surfactants are alkyl polyglucosides having 8 to 22, preferably 10 to 18, carbon atoms in the alkyl chain. These compounds contain at most 1 to 20, preferably 1.1 to 5, glucoside units.

Another class of nonionic surfactants are N-alkyl-glucamides of the structure II or III

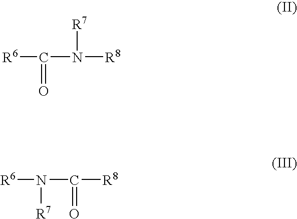

in which R⁶ is C₆-C₂₂-alkyl, R⁷ is H or C₁-C₄-alkyl and R⁸ is a polyhydroxyalkyl radical having 5 to 12 carbon atoms and at least 3 hydroxyl groups. Preferably, R₆ is C₁₀-C₁₈-alkyl, R⁷ is methyl and R⁸ is a C₅-C₆-radical. Such compounds are obtained, for example, by the acylation of reductively aminated sugars with acid chlorides of C₁₀-C₁₈-carboxylic acids.

### Organic Cobuilders

Examples of suitable low molecular weight polycarboxylates as organic cobuilders are: C₄-C₂₀-di-, -tri- and -tetracarboxylic acids, such as, for example, succinic acid, propanetricarboxylic acid, butanetetracarboxylic acid, cyclopentanetetra-carboxylic acid and alkyl- and alkenylsuccinic acids having C₂-C₁₆-alkyl or -alkenyl radicals;

C₄-C₂₀-hydroxycarboxylic acids, such as, for example, malic acid, tartaric acid, gluconic acid, glucaric acid, citric acid, lactobionic acid and sucrose mono-, -di- and -tricarboxylic acid;

aminopolycarboxylates, such as, for example, nitrilo-triacetic acid, methylglycinediacetic acid, alaninediacetic acid, ethylenediaminetetraacetic acid and serinediacetic acid;

salts of phosphonic acids, such as, for example, hydroxyethanediphosphonic acid, ethylenediaminetetra(methylenephosphonate) and diethylenetriaminepenta-(methylenephosphonate).

Examples of suitable oligomeric or polymeric polycarboxylates as organic cobuilders are:

oligomaleic acids, as described, for example, in EP-A-451 508 and EP-A-396 303;

co- and terpolymers of unsaturated C₄-C₈-dicarboxylic acids, where, as comonomers, monoethylenically unsaturated monomers

from group (i) in amounts of up to 95% by weight

from group (ii) in amounts of up to 60% by weight

from group (iii) in amounts of up to 20% by weight

may be present in copolymerized form.

Examples of suitable unsaturated C₄-C₈-dicarboxylic acids are, for example, maleic acid, fumaric acid, itaconic acid and citraconic acid. Preference is given to maleic acid.

The group (i) includes monoethylenically unsaturated C₃-C₈-monocarboxylic acids, such as, for example, acrylic acid, methacrylic acid, crotonic acid and vinyl acetic acid. Preference is given to using acrylic acid and methacrylic acid from group (i).

The group (ii) includes monoethylenically unsaturated C₂-C₂₂-olefins, vinyl alkyl ethers having C₁-C₈-alkyl groups, styrene, vinyl esters of C₁-C₈ carboxylic acids, (meth)acrylamide and vinylpyrrolidone. Preference is given to using C₂-C₆-olefins, vinyl alkyl ethers having C₁-C₄-alkyl groups, vinyl acetate and vinyl propionate from group (ii).

The group (iii) includes (meth)acrylic esters of C₁-C₈-alcohols, (meth)acrylonitrile, (meth)acrylamides of C₁-C₈-amines, N-vinylformamide and vinylimidazole.

If the polymers of group (ii) contain vinyl esters in copolymerized form, these may also be present partly or completely in hydrolyzed form to give vinyl alcohol structural units. Suitable co- and terpolymers are known, for example, from U.S. Pat. No. 3,887,806 and DE-A 43 13 909.

As copolymers of dicarboxylic acids, suitable organic cobuilders are preferably:

copolymers of maleic acid and acrylic acid in the weight ratio 10:90 to 95:5, particularly preferably those in the weight ratio 30:70 to 90:10 having molar masses of from 10 000 to 150 000;

terpolymers of maleic acid, acrylic acid and a vinyl ester of a C₁-C₃-carboxylic acid in the weight ratio 10(maleic acid):90(acrylic acid+vinyl ester) to 95(maleic acid):5(acrylic acid+vinyl ester), where the weight ratio of acrylic acid to vinyl ester can vary in the range from 20:80 to 80:20, and particularly preferably

terpolymers of maleic acid, acrylic acid and vinyl acetate or vinyl propionate in the weight ratio 20(maleic acid):80(acrylic acid+vinyl ester) to 90(maleic acid):10(acrylic acid+vinyl ester), where the weight ratio of acrylic acid to the vinyl ester can vary in the range from 30:70 to 70:30;

copolymers of maleic acid with C₂-C₈-olefins in the molar ratio 40:60 to 80:20, where copolymers of maleic acid with ethylene, propylene or isobutane in the molar ratio 50:50 are particularly preferred.

Graft polymers of unsaturated carboxylic acids to low molecular weight carbohydrates or hydrogenated carbohydrates, cf. U.S. Pat. No. 5,227,446, DE-A-44 15 623, DE-A-43 13 909, are likewise suitable as organic cobuilders.

Examples of suitable unsaturated carboxylic acids in this connection are maleic acid, fumaric acid, itaconic acid, citraconic acid, acrylic acid, methacrylic acid, crotonic acid and vinyl acetic acid, and mixtures of acrylic acid and maleic acid which are grafted on in amounts of from 40 to 95% by weight, based on the component to be grafted. For the modification, it is additionally possible for up to 30% by weight, based on the component to be grafted, of further monoethylenically unsaturated monomers to be present in copolymerized form. Suitable modifying monomers are the abovementioned monomers of groups (ii) and (iii).

Suitable graft bases are degraded polysaccharides, such as, for example, acidic or enzymatically degraded starches, inulins or cellulose, reduced (hydrogenated or reductively aminated) degraded polysaccharides, such as, for example, mannitol, sorbitol, aminosorbitol and glucamine, and also polyalkylene glycols having molar masses up to M_{w}=5 000, such as, for example, polyethylene glycols, ethylene oxide/propylene oxide or ethylene oxide/butylene oxide block copolymers, random ethylene oxide/propylene oxide or ethylene oxide/butylene oxide copolymers, alkoxylated mono- or polybasic C₁-C₂₂alcohols, cf. U.S. Pat. No. 4,746,456.

From this group, preference is given to using grafted degraded or degraded reduced starches and grafted polyethylene oxides, in which case 20 to 80% by weight of monomers, based on the graft component, are used in the graft polymerization. For the grafting, preference is given to using a mixture of maleic acid and acrylic acid in the weight ratio from 90:10 to 10:90.

Polyglyoxylic acids as organic cobuilders are described, for example, in EP-B-001004, U.S. Pat. No. 5,399,286, DE-A-4106 355 and EP-A-656 914. The end-groups of the polyglyoxylic acids may have different structures.

Polyamidocarboxylic acids and modified polyamidocarboxylic acids as organic cobuilders are known, for example, from EP-A-454 126, EP-B-511037, WO-A 94/01486 and EP-A-581 452.

As organic cobuilders, preference is also given to using polyaspartic acid or cocondensates of aspartic acid with further amino acids, C₄-C₂₅-mono- or -dicarboxylic acids and/or C₄-C₂₅-mono- or -diamines. Particular preference is given to using polyaspartic acids prepared in phosphorus-containing acids and modified with C₆-C₂₂-mono- or -dicarboxylic acids or with C₆-C₂₂-mono- or -diamines.

Condensation products of citric acid with hydroxycarboxylic acids or polyhydroxy compounds as organic cobuilders are known, for example, from WO-A 93/22362 and WO-A 92/16493. Such carboxyl-containing condensates usually have molar masses up to 10 000, preferably up to 5 000.

### Antiredeposition Agents and Soil Release Polymers

Suitable soil release polymers and/or antiredeposition agents for detergents are, for example:

polyesters of polyethylene oxides with ethylene glycol and/or propylene glycol and aromatic dicarboxylic acids or aromatic and aliphatic dicarboxylic acids;

polyesters of polyethylene oxides terminally capped at one end with di- and/or polyhydric alcohols and dicarboxylic acid.

Such polyesters are known, for example from U.S. Pat. No. 3,557,039, GB-A 1 154 730, EP-A-185 427, EP-A-241 984, EP-A-241 985, EP-A-272 033 and U.S. Pat. No. 5,142,020.

Further suitable soil release polymers are amphiphilic graft or copolymers of vinyl and/or acrylic esters on polyalkylene oxides (cf. U.S. Pat. No. 4,746,456, U.S. Pat. No. 4,846,995, DE-A-37 11 299, U.S. Pat. No. 4,904,408, U.S. Pat. No. 4,846,994 and U.S. Pat. No. 4,849,126) or modified celluloses, such as, for example, methylcellulose, hydroxypropylcellulose or carboxymethylcellulose.

### Color-transfer Inhibitors

Examples of the color-transfer inhibitors used are homo- and copolymers of vinylpyrrolidone, vinylimidazole, vinyloxazolidone and 4-vinylpyridine N-oxide having molar masses of from 15 000 to 100 000, and crosslinked finely divided polymers based on these monomers. The use mentioned here of such polymers is known, cf. DE-B-22 32 353, DE-A-28 14 287, DE-A-28 14 329 and DE-A-43 16 023.

### Enzymes

Suitable enzymes are, for example, proteases, amylases, lipases and cellulases, in particular proteases. It is possible to use two or more enzymes in combination.

In addition to use in detergents and cleaners for the domestic washing of textiles, the detergent compositions which can be used according to the invention can also be used in the sector of commercial textile washing and of commercial cleaning. In this field of use, peracetic acid is usually used as bleach, which is added to the wash liquor as an aqueous solution.

### Use in Textile Detergents

A typical pulverulent or granular heavy-duty detergent according to the invention may, for example, have the following composition:

0.5 to 50% by weight, preferably 5 to 30% by weight, of at least one anionic and/or nonionic surfactant,

0.5 to 60% by weight, preferably 15 to 40% by weight, of at least one inorganic builder,

0 to 20% by weight, preferably 0.5 to 8% by weight, of at least one organic cobuilder,

2 to 35% by weight, preferably 5 to 30% by weight, of an inorganic bleach,

0.1 to 20% by weight, preferably 0.5 to 10% by weight, of a bleach activator, optionally in a mixture with further bleach activators,

0 to 1% by weight, preferably up to at most 0.5% by weight, of a bleach catalyst,

0 to 5% by weight, preferably 0 to 2.5% by weight, of a polymeric color-transfer inhibitor,

0 to 1.5% by weight, preferably 0.1 to 1.0% by weight, of protease,

0 to 1.5% by weight, preferably 0.1 to 1.0% by weight, of lipase,

0 to 1.5% by weight, preferably 0.2 to 1.0% by weight, of a soil release polymer,

ad 100% with customary auxiliaries and adjuncts and water.

Inorganic builders preferably used in detergents are sodium carbonate, sodium hydrogen carbonate, zeolite A and P, and amorphous and crystalline Na silicates.

Organic cobuilders preferably used in detergents are acrylic acid/maleic copolymers, acrylic acid/maleic acid/vinyl ester terpolymers and citric acid.

Inorganic bleaches preferably used in detergents are sodium perborate and sodium carbonate perhydrate.

Anionic surfactants preferably used in detergents are the novel linear and slightly branched alkylbenzenesulfonates (LAS), fatty alcohol sulfates and soaps.

Nonionic surfactants preferably used in detergents are C₁₁-C₁₇-oxo alcohol ethoxylates having 3-13 ethylene oxide units, C₁₀-C₁₆-fatty alcohol ethoxylates having 3-13 ethylene oxide units, and ethoxylated fatty alcohols or oxo alcohols additionally alkoxylated with 1-4 propylene oxide or butylene oxide units.

Enzymes preferably used in detergents are protease, lipase and cellulase. Of the commercially available enzymes, amounts of from 0.05 to 2.0% by weight, preferably 0.2 to 1.5% by weight, of the formulated enzyme, are generally added to the detergent. Suitable proteases are, for example, Savinase, Desazym and Esperase (manufacturer: Novo Nordisk). A suitable lipase is, for example, Lipolase (manufacturer: Novo Nordisk). A suitable cellulase is, for example, Celluzym (manufacturer: Novo Nordisk).

Soil release polymers and antiredeposition agents preferably used in detergents are graft polymers of vinyl acetate on polyethylene oxide of molecular mass 2 500-8 000 in the weight ratio 1.2:1 to 3.0:1, polyethylene terephthalates/oxyethylene terephthalates of molar mass 3 000 to 25 000 from polyethylene oxides of molar mass 750 to 5 000 with terephthalic acid and ethylene oxide and a molar ratio of polyethylene terephthalate to polyoxyethylene terephthalate of from 8:1 to 1:1, and block polycondensates according to DE-A-44 03 866.

Color-transfer inhibitors preferably used in detergents are soluble vinylpyrrolidone and vinylimidazole copolymers having molar masses greater than 25 000, and finely divided crosslinked polymers based on vinylimidazole.

The pulverulent or granular detergents according to the invention can comprise up to 60% by weight of inorganic extenders. Sodium sulfate is usually used for this purpose. However, the detergents according to the invention preferably have a low content of extenders and comprise only up to 20% by weight, particularly preferably only up to 8% by weight, of extenders.

The detergents according to the invention can have various bulk densities in the range from 300 to 1 200 g/l, in particular 500 to 950 g/l. Modem compact detergents generally have high bulk densities and exhibit a granular structure.

The invention is described in more detail by reference to the examples below.

### EXAMPLE 1

A butadiene-free C₄ fraction with a total butene content of 84.2% by weight and a 1-butene to 2-butene molar ratio of 1 to 1.06 is passed continuously at 40° C. and 10 bar over a tubular reactor fitted with Re₂O₇/Al₂O₃ heterogeneous catalyst. The space velocity in the example is 4 500 kg/m²h. The reaction discharge is separated by distillation and comprises the following components (data in percent by mass):

ethene 1.15%; propene 18.9%, butanes 15.8%, 2-butenes 19.7%, 1-butene 13.3%, i-butene 1.0%, 2-pentene 19.4%, methylbutene 0.45%, 3-hexene 10.3%. 2-Pentene and 3-hexene are isolated from the product by distillation in purities of >99% by weight.

### EXAMPLE 2

Continuous dimerization of 3-hexene in the fixed-bed process

Catalyst: 50% NiO, 34% SiO₂, 13% TiO₂, 3% Al₂O₃ (as in DE 43 39 713) used as 1-1.5 mm chips (100 ml), conditioned for 24 h at 160° C. in N₂

Reactor: isothermal, 16 mm Ø reactor

WHSV: 0.25 kg/1.h

Pressure: 20 to 25 bar

Temperature: 100 to 160° C.

The collative product was distilled to a C₁₂ purity of 99.9% by weight, and a determination of the skeletal isomers of the C₁₂ fraction was carried out (14.2% n-dodecenes, 31.8% 5-methylundecenes, 29.1% 4-ethyldecenes, 6.6% 5,6-dimethyldecenes, 9.3% 4-methyl-5-ethylnonene 3.7% 4,5-diethyloctenes, percentages are by weight).

### EXAMPLE 3

2-Pentene from the raffinate II metathesis was dimerized continuously as in example 2 over an Ni heterogeneous catalyst. Fractional distillation of the product gave a decene fraction with a purity of 99.5%. ¹H NMR spectroscopy was used after hydrogenation to determine an isoindex of 1.36. The hydrogenated sample was then analyzed with regard to the skeletal isomers of the paraffins using gas chromatography. (n-Decane 13.0%, 4-methylnonane 26.9%, 3-ethyloctane 16.5%, 4,5-dimethyloctane 5.4%, 3,4-diethylhexane 6.8%, 3-ethyl-4-methylheptane 9.2%, (the percentages are by weight)). The sample contains 22% C10 paraffins of a structure which cannot be assigned.

### EXAMPLE 4

A mixture of 2-pentene and 3-hexene from the raffinate II methathesis was dimerized as in example 2 and example 3. Fractional distillation of the product gave a decene/undecene/dodecene fraction with a purity of 99.5%

### EXAMPLE 5

### Comparison

A 6 l reactor was charged with 6 458 g of benzene and 39.2 g of AlCl₃ and, with stirring, 1393 g of a C₁₂-olefin mixture corresponding to example 2 were metered in. The reaction temperature of 20° C. was regulated by cooling in an ice bath and by varying the metering rate of the olefin mixture. After 55 min, the reaction mixture was decanted, neutralized with NaOH and washed with demineralized water. Filtration and drying over round and cotton wool filters was then carried out. The LAB yield was 83.4%. The alkylbenzene mixture consisted of 56% PhCHRR′, 44% PhCRR′R″ and 0% PhCH₂R.

### EXAMPLE 6

A 2 l four-necked flask fitted with magnetic stirrer, thermometer, dropping funnel, gas inlet frit and gas outlet is charged with 1 900 g of SO₃-depleted oleum. This flask is connected via the gas outlet to a 11 three-necked flask via a Viton hose.

This 1 l flask fitted with paddle stirrer, thermometer, gas inlet frit and gas outlet is charged with an alkylbenzene mixture analogously to example 5.

The depleted oleum is brought to 120° C. in the SO₃-developer, and the oleum (65% strength) is added via a dropping funnel over the course of 30 minutes. Using a stream of nitrogen of 80 l/h, the SO₃ gas is stripped out and passed into the alkylbenzene via a 6 mm inlet tube. The temperature of the alkylbenzene/alkylbenzenesulfonic acid mixture increases slowly to 40° C. and is maintained at 40° C. using cooling water. The residual gas is removed by suction using a water-jet pump.

The molar ratio of SO₃/alkylbenzene is 1.01:1.

After a postreaction time of 4 h, the alkylbenzene-sulfonic acid formed is stabilized with 0.4% by weight of water and then neutralized with NaOH to give the alkylbenzenesulfonate.

### EXAMPLE 7

12.75 g of HY zeolite (Si:Al=5.58:1 molar) were dried at 500° C. for 5 h and stirred together with 120 g of benzene, 25.5 g of a C₁₂-olefin mixture corresponding to example 2 in a 300 ml steel autoclave for 6 h at 180° C. under N₂. The zeolite was then separated off, and the product mixture was analyzed using GC (column DB-5, 50 m). It consisted of 87.1% benzene, 3.7% unreacted C₁₂-olefin, 7.6% dodecylbenzene and <0.1% heavy alkylate (dialkylbenzenes) in addition to small amounts of unidentified hydrocarbons. The product mixture was distilled under reduced pressure at 1 mbar. Between 130° C. and 150° C., 9.5 g of an alkylbenzene mixture consisting of 97% PhCHRR′, 0% PhCRR′R″ and 3% PhCH₂R were obtained.

### EXAMPLE 8

An alkylbenzene mixture analogous to example 7 was reacted to give the alkylbenzene sulfonate as detailed in example 6.

### EXAMPLE 9

### Comparison

12.75 g of H-MOR zeolite (Si:Al=24.5:1 molar) were dried at 500° C. for 5 h and stirred together with 120 g of benzene, 25.5 g of a C₁₂-olefin mixture corresponding to example 2 in a 300 ml steel autoclave for 6 h at 180° C. under N₂. The zeolite was then separated off, and the product mixture was analyzed using GC (column DB-5, 50 m). It consisted of 85.1% benzene, 8.8% unreacted C₁₂-olefin, 4.4% dodecylbenzene and <0.1% heavy alkylate (dialkylbenzenes) in addition to small amounts of unidentified hydrocarbons. The product mixture was distilled under reduced pressure at 1 mbar. Between 130° C. and 150° C., 4.9 g of an alkylbenzene mixture consisting of 96% PhCHRR′, 2% PhCRR′R″ and 2% PhCH₂R were obtained.

### EXAMPLE 10

### Comparison

15 12.75 g of H-ZSM-5 zeolite (Si:Al=42.5:1 molar) were dried at 500° C. for 5 h and stirred together with 120 g of benzene, 25.5 g of a C₁₂-olefin mixture corresponding to example 2 in a 300 ml steel autoclave for 6 h at 1 80° C. under N₂. The zeolite was then separated off, and the product mixture was analyzed by means of GC (column DB-5, 50 m). It consisted of 88.6% benzene, 7.1% unreacted C₁₂-olefin, 1.0% dodecylbenzene and <0.1% heavy alkylate (dialkylbenzenes) in addition to small amounts of unidentified hydrocarbons.

### EXAMPLE 11

### Comparison

12.75 g of H-MCM-22 zeolite (Si:Al=18.8:1 molar) were dried at 500° C. for 5 h and stirred together with 120 g of benzene, 25.5 g of a C₁₂-olefin mixture corresponding to example 2 in a 300 ml steel autoclave for 6 h at 180° C. under N₂. The zeolite was then separated off, and the product mixture was analyzed by means of GC (column DB-5, 50 m). It consisted of 87.1% benzene, 5.6% unreacted C₁₂-olefin, 6.7% dodecylbenzene and <0.1% heavy alkylate (dialkylbenzenes) in addition to small amounts of unidentified hydrocarbons.

The product mixture was distilled under reduced pressure at 1 mbar. Between 130° C. and 150° C., 8.4 g of an alkylbenzene mixture consisting of 73% PhCHRR′, 23% PhCRR′R″ and 4% PhCH₂R were obtained.

### EXAMPLE 12

12.75 g of HY zeolite (Si:Al=5.58:1 molar) were dried at 500° C. for 5 h and stirred together with 120 g of benzene, 25.5 g of a C₁₀-olefin mixture corresponding to example 3 in a 300 ml steel autoclave for 6 h at 180° C. under N₂. The zeolite was then separated off, and the product mixture was analyzed by means of GC (column DB-5, 50 m). The product displayed the following isomer distribution: 96% PhCHRR′, 0% PhCRR′R″ and 4% PhCH₂R.

### EXAMPLE 13

An alkylbenzene mixture analogous to example 12 was reacted to give the alkylbenzenesulfonate as detailed in example 6.

### EXAMPLE 14

12.75 g of HY zeolite (Si:Al=5.58:1 molar) were dried for 5 h at 500° C. and stirred together with 120 g of benzene, 25.5 g of a C₁₀₋₁₂-olefin mixture corresponding to example 4 in a 300 ml steel autoclave for 6 h at 180° C. under N₂. The zeolite was then separated off, and the product mixture was analyzed by means of GC (column DB-5, 50 m). The product displayed the following isomer distribution: 97% PhCHRR′, 1% PhCRR′R″ and 2% PhCh₂R.

### EXAMPLE 15

An alkylbenzene mixture analogous to example 14 was reacted to give the alkylbenzene sulfonate as detailed in example 6.

### EXAMPLE 16

1 l/h of oleum (65%) in concentrated sulfuric acid is introduced into a heated (120° C.) 10 l four-necked flask using a pump. 130 l/h of dry air are passed through the sulfuric acid via a frit; this air strips out the SO₃. The SO₃-enriched stream of air (about 4% of S₃) is brought into contact with an alkylbenzene mixture from example 13 in a 2 m-long fallingfilm reactor, at approximately 40-50° C. (10-15° C. jacket water cooling), and sulfonates this mixture. The molar ratio of SO₃/alkylbenzene is 1.01:1. The reaction time in the falling-film reactor is approximately 10 sec. The product is pumped to an afterripening container where it remains for approximately 4-8 h. The sulfonic acid is then stabilized with 0.4% by weight of water and neutralized with NaOH to give the alkylbenzenesulfonate.

We claim:

1. A process for the preparation of alkylaryl compounds by
1) preparation of a mixture of, on statistical average, predominantly monobranched C₁₀₋₁₄-olefins by
a) reaction of a C₄-olefin mixture over a metathesis catalyst for the preparation of an olefin mixture comprising 2-pentene and/or 3-hexene, and optional removal of 2-pentene and/or 3-hexene, followed by dimerization of the resulting 2-pentene and/or 3-hexene over a dimerization catalyst to give a mixture comprising C₁₀₋₁₂-olefins, and optionally removal of the C₁₀₋₁₂-olefins, or
b) extraction of predominantly monobranched paraffins from kerosene cuts and subsequent dehydrogenation, or
c) Fischer-Tropsch synthesis of olefins or paraffins, where the paraffins are dehydrogenated, or
d) dimerization of shorter-chain internal olefins, or
e) isomerization of linear olefins or paraffins, where the isomerized paraffins are dehydrogenated,
2) reaction of the olefin mixture obtained in stage 1) with an aromatic hydrocarbon in the presence of an alkylation catalyst which contains zeolites of the faujasite type.

2. A process for the preparation of alkylarylsulfonates by preparing alkylaryl compounds as claimed in claim 1 and subsequently
3) sulphonation and neutralization of the alkylaryl compounds obtained in stage 2).

3. A process as claimed in claim 1 or 2, wherein in stage 1 a) the metathesis catalyst is chosen from compounds of a metal of transition groups VIb, VIIb or VIII of the Periodic Table of the Elements.

4. A process as claimed in any of claims 1 to 3, wherein, in stage 2), the reaction conditions and the catalyst are chosen such that the resulting alkylaryl compounds in the alkyl radical have 1 to 3 carbon atoms with an H/C index of 1, and the proportion of carbon atoms with an H/C index of 0 in the alkyl radical is statistically less than 5%.

5. An alkylaryl compound obtainable by the process as claimed in claim 1.

6. An alkylarylsulfonate obtainable by the process as claimed in claim 2.

7. The use of an alkylarylsulfonate as claimed in claim 6 as surfactant.

8. The use as claimed in claim 7 in detergents and cleaners.

9. A detergent or cleaner comprising, in addition to customary ingredients, an alkylarylsulfonate as claimed in claim 6.
